(19) United States
(12) Patent Application Publication
(10) Pub. No.: US 2024/0285493 A1
(43) Pub. Date: Aug. 29, 2024

(54) COSMETIC PRODUCT CONSISTING OF PACKAGING AND A PREPARATION CONTAINING PROPYLENE GLYCOL DIESTER

(71) Applicant: BEIERSDORF AG, Hamburg (DE)

(72) Inventors: Claudia MUELLER, Tangstedt (DE); Jette Mareike NEBEN, Hamburg (DE); Sabrina DIEKERT, Hamburg (DE); Astrid ALBRECHT, Ellerbek (DE)

(21) Appl. No.: 18/568,375

(22) PCT Filed: Jun. 9, 2022
(86) PCT No.: PCT/EP2022/065646

§ 371 Date: Dec. 8, 2023

(30) Foreign Application Priority Data
Jun. 14, 2021 (DE) 10 2021 206 043.9

(51) Int. Cl. A61K 8/37; A45D 34/00; A61K 8/06; A61Q 19/00

(52) U.S. Cl. CPC A61K 8/375; A45D 34/00; A61K 8/062; A61K 8/064; A61Q 19/00; A61K 2800/10; A61K 2800/30; A61K 2800/34; A61K 2800/87

(57) ABSTRACT

Cosmetic product of a) a packaging of high-density polyethylene (HDPE), low-density polyethylene (LDPE), polypropylene (PP) or polyethylene terephthalate (PET), and b) a cosmetic preparation containing propylene glycol diester.

The present invention relates to a cosmetic product of a cosmetic preparation containing propylene glycol diester and a packaging of HDPE, LDPE, PP or PET.

The desire to look beautiful and attractive is naturally rooted in humans. Although ideals of beauty have changed over time, the pursuit of a flawless appearance has always been aimed for by humans. An essential part of a beautiful and attractive appearance is the condition and complexion of the skin.

In order for the skin to be able to perform the full range of its biological functions, it requires regular cleansing and care. Cleansing of the skin serves to remove dirt, sweat and residual dead skin particles, which form an ideal nutrient source for all kinds of germs and parasites. Skincare products mostly serve for moisturizing and refatting the skin. Active ingredients are commonly added thereto, which are intended to regenerate the skin and for example to prevent and reduce the premature aging thereof (for example the appearance of fine lines and wrinkles).

Skincare products are generally stored in packagings in the form of bottles, tubes or jars. These predominantly consist of glass (glass jars) or plastics such as high-density polyethylene (HDPE), low-density polyethylene (LDPE), polypropylene (PP) or polyethylene terephthalate (PET).

A disadvantage of such packagings, which are inherently very favorable and extremely hygienic, is the fact that these packagings are not particularly sustainable/environmentally friendly, since they are produced from petroleum products and/or natural gas and ultimately release carbon dioxide from fossil sources during their thermal utilization (incineration in a waste incineration plant).

In order to conserve natural resources, attempts are therefore being made to produce these packagings as thin-walled as possible and to recycle them after the preparation has been used.

However, these thin-walled packagings have the disadvantage that, if the cosmetic preparation with which they are filled comprises one or more oil components, the packagings are not particularly storage-stable but rather swell on contact with the oil, since the oil is “soaked up” and absorbed by the plastic. Moreover, oil may separate out of the products. Furthermore, the oil in the plastic may cause printed labelling on the packaging to smudge and rub off. In addition, the oil absorbed by the packaging makes it difficult to recycle the plastic because the oil has to be laboriously separated from the polymer before a new packaging can be produced again from the polymer plastic. For these reasons, either only oil-free preparations (e.g. toothpaste) can be used to fill conventional, thin-walled packagings, or the packaging must be elaborately coated or sealed on the inner side facing the cosmetic product. Known from the prior art, for example, are protective barriers made of ethylene vinyl alcohol, which are relatively expensive and also less easy to recycle than pure polyolefin packagings.

The problem of the absorption of oils from the preparation by the packaging is further exacerbated by the general trend to ban certain oil components from industrial preparation of cosmetic preparations, for example due to their poor degradability or high emission levels during production. Dimethicone in particular was/is particularly popular with product developers per se due to its low tendency to migrate into plastic packagings. On the other hand, there are various reasons why cosmetic preparations should be formulated without dimethicone.

It was therefore an object of the present invention to reduce and ideally to eliminate the disadvantages of the prior art and to develop a cosmetic product composed of an oil-containing cosmetic preparation and a plastic packaging, the packaging of which is easier to recycle and is storage-stable, i.e. in which oil absorption by the plastic packaging is significantly reduced, without it being necessary to apply a protective layer (e.g., of ethylene vinyl alcohol) to the plastic.

The object was surprisingly achieved by a cosmetic product of
a) a packaging of high-density polyethylene (HDPE), low-density polyethylene (LDPE), polypropylene (PP) or polyethylene terephthalate (PET), and
b) a cosmetic preparation containing propylene glycol diester.

Although those skilled in the art are familiar with cosmetic products having packagings of HDPE, PP or PET in which cosmetic preparations with ester oils, such as C12-15 alkyl benzoate, or ether oils, such as dicaprylyl ether, are present, these oil components of the prior art tend to migrate relatively heavily into these packaging materials. Therefore, such products usually have a protective layer made of, for example, ethylene vinyl alcohol on the inner side, which is usually not declared or does not have to be declared on the packaging.

It is preferred according to the invention if the packaging a) is formed from high-density polyethylene (HDPE).

Furthermore, it is advantageous according to the invention if the packaging a) has a wall thickness of 1 to 8 mm.

Moreover, the embodiments of the present invention that are advantageous according to the invention are characterized in that the packaging does not have any protective layer (for example made of ethylene vinyl alcohol) on the inner side facing the preparation.

Embodiments of the present invention that are advantageous according to the invention are characterized in that one or more compounds from the group propylene glycol diheptanoate (INCI: Propylene Glycol Diheptanoate), propylene glycol dicaprate (INCI: Propylene Glycol Dicaprate), propylene glycol dicaprylate/dicaprate (INCI: Propylene Glycol Dicaprylate/Dicaprate) and propylene glycol dibenzoate (INCI: Propylene Glycol Dibenzoate). are used as propylene glycol diesters.

In this case, it is preferred according to the invention if propylene glycol diheptanoate (INCI: Propylene Glycol Diheptanoate) is used as propylene glycol diester.

Embodiments of the present invention that are advantageous according to the invention are also obtained in that the preparation is free of dimethicone (INCI: Dimethicone).

Moreover, it is advantageous according to the invention if the preparation is free of silicone oil, mineral oil, paraffin wax, microcrystalline wax, shellac wax and polyethylene waxes.

It is also advantageous according to the invention if the preparation is free of polyacrylates, crosslinked acrylate/C10-C30 alkyl acrylate polymers and vinylpyrrolidone/hexadecene copolymers, and also free of 3-(4-methylbenzylidene)camphor, 2-hydroxy-4-methoxybenzophenone (INCI: Oxybenzone), 2-ethylhexyl 4-methoxycinnamate (INCI: Octyl Methoxycinnamate), ethylhexyl 2-cyano-3,3-diphenylacrylate (INCI: Octocrylene), parabens (particularly methyl, propyl and butyl paraben), methylisothiazolinone, chloromethylisothiazolinone and DMDM hydantoin, polyethylene glycol ethers or polyethylene glycol esters.

It is also advantageous in the sense of the present invention if the cosmetic preparation is in the form of an emulsion. It is preferred according to the invention here if the cosmetic preparation is in the form of an oil-in-water emulsion (O/W emulsion).

It is advantageous according to the invention if the cosmetic preparation comprises propylene glycol diesters at a concentration of 0.1% to 10% by weight, based on the total weight of the preparation. Preferred according to the invention here is a use concentration of 0.1% to 5% by weight, based on the total weight of the preparation. These use concentrations refer to the total amount of propylene glycol diesters used. If only one propylene glycol diester (e.g. propylene glycol diheptanoate) is used, this is the advantageous or preferred use concentration for this one compound.

It is also advantageous according to the invention if the total amount of propylene glycol diesters in the oil phase of the preparation is at least 3% by weight, based on the total weight of the oil phase.

Embodiments that are advantageous according to the invention are also characterized in that the preparation contains one or more oils selected from the group of the compounds octyldodecanol, caprylic/capric triglyceride (INCI: Caprylic/Capric Triglyceride), cocoglycerides (INCI: Cocoglycerides), tridecyl stearate (INCI: Tridecyl Stearate), tridecyl trimellitate (INCI: Tridecyl Trimellitate).

Furthermore, it is advantageous according to the invention if the preparation contains one or more oils selected from the group of the compounds polycitronellol (INCI: Polycitronellol), polycitronellol acetate (INCI: Polycitronellol Acetate), triolein, ethylhexyl acetoxystearate (INCI: Ethylhexyl Acetoxystearate), acetyl ethylhexyl polyhydroxystearate (INCI: Acetyl Ethylhexyl Polyhydroxystearate).

It is also advantageous according to the invention if the preparation contains one or more oils selected from the group of the compounds almond oil (INCI: Prunus Amygdalus Dulcis Oil) and/or sunflower oil (INCI: Helianthus Annuus Seed Oil), hydrogenated castor oil (INCI: Hydrogenated Castor Oil).

Furthermore, it is advantageous according to the invention if the preparation contains tocopherol. Otherwise, the preparation according to the invention may comprise the customary ingredients for cosmetics and be composed accordingly.

### Comparative Experiments

The penetration of the following cosmetic oils into the HDPE packaging material was investigated. In this case, the product “Hostalen ACP 5831D” from Lyondellbasell was used as HDPE.

### Test Procedure

1. Use three die-cuts from the packaging material having a diameter of 5 mm and a height of 1 mm for each raw material/each cosmetic preparation.
2. Determine the initial weight of each die-cut on an analytical balance. (Initial weight=m0)
3. Place the die-cuts in a sealable container and add the raw material/cosmetic preparation. The die-cuts must be completely enveloped by the raw material/cosmetic preparation.
4. Store the closed container in an incubator at 40±1° C.
5. Mechanically clean the die-cuts with cellulose after 28 days.
5. Reweigh the die-cuts after 28 days.

| Weight of the sample | |
|---|---|
| (die-cut) | Determination |
| m0 | Initial weight |
| m1 | 28 days |

### Note

Do not touch the die-cuts with your fingers; always use tweezers to avoid contamination.

### Evaluation

Raw materials or constituents of the cosmetic preparation have migrated into the packaging material if the weight of the die-cut m1 is greater than the weight of m0.

$$Weight increase ( \% ) = ( m 1 - m 0 ) / m 0$$

Using the formula (m1−m0)/m0, the weight increase (%) due to the raw material or the cosmetic preparation which has migrated into the packaging material is determined.

The mean value [1%] and standard deviation [1%] of the triple determination are also calculated.

### RESULTS

| | Weight increase in % after 28 d | |
|---|---|---|
| | Mean | Standard deviation |
| INCI | [%] | [%] |
| Propylene Glycol Diheptanoate | 3.18 | 0.01 |
| Propylene Glycol Dicaprate | 3.34 | 0.01 |
| Propylene Glycol | 3.30 | 0.01 |
| Dicaprylate/Dicaprate | | |
| Propylene Glycol Dibenzoate | 1.16 | 0.05 |
| Paraffinum Liquidum | 5.24 | 0.05 |
| C12-15 Alkyl benzoate | 4.85 | 0.04 |
| Dicaprylyl ether | 5.52 | 0.04 |

### EXAMPLES

The examples below are intended to elucidate the present invention without limiting it. Unless otherwise stated, all amounts, proportions, and percentages are based on the weight and total amount and on the total weight of the preparations.

| | O/W Lotion | W/O Lotion |
|---|---|---|
| INCI | % by weight | % by weight |
| Propylene Glycol Diheptanoate | 3.5 | 5 |
| Ethylhexylglycerin | 0.2 | 0 |
| Butyrospermum Parkii Butter | 1.2 | 1 |
| Cetyl Palmitate | 0 | 0.6 |
| Isopropyl Palmitate | 0 | 5 |
| Cocoglycerides | 0 | 5 |
| Coco-Caprylate/Caprate | 0 | 5 |
| Sodium Cetearyl Sulfate | 0.1 | 0 |
| Glyceryl Stearate SE | 2.2 | 0 |
| Diisostearoyl Polyglyceryl-3 Dimer Dilinoleate | 0 | 1.4 |
| Polyglyceryl-4 Diisostearate/Polyhydroxystearate/Sebacate | 0 | 0.8 |
| Glycerin | 7 | 7.5 |
| Citric Acid | 0 | 0.09 |
| Sodium Citrate + Aqua | 0 | 0.175 |
| Phenoxyethanol | 1 | 0 |
| Potassium Sorbate | 0 | 0.13 |
| Cetearyl Alcohol | 4.5 | 0 |
| Xanthan Gum | 0.1 | 0 |
| Hydroxypropyl Starch Phosphate + Aqua | 0.5 | 0 |
| Magnesium Sulfate | 0 | 0.7 |
| Aqua | ad 100 | ad 100 |

These preparations were filled and stored in a bottle-shaped storage container made of HDPE (Hostalen ACP 5831 D, from Lyondellbasell).

### Further Example Formulations

### Example 1: O/W Lotion

| | % by |
|---|---|
| INCI | weight |
| Propylene Glycol Diheptanoate | 3.5 |
| Ethylhexylglycerin | 0.2 |
| Sodium Cetearyl Sulfate | 0.1 |
| Glyceryl Stearate SE | 2 |
| Glycerin | 5 |
| Benzyl Alcohol | 0.2 |
| Cetearyl Alcohol | 5 |
| Helianthus Annuus Seed Oil | 0.1 |
| Xanthan Gum | 0.1 |
| Alcohol Denat. | 5 |
| Hydroxypropyl Starch Phosphate + Aqua | 1.3 |
| Citric Acid (pH: 6.5) | qs |
| Aqua | ad 100 |

### Example 2: W/O Lotion

| INCI | % by weight |
|---|---|
| Propylene Glycol Diheptanoate | 3 |
| Butyrospermum Parkii Butter | 1 |
| Cetyl Palmitate | 0.6 |
| Isopropyl Palmitate | 8 |
| Vegetable Oil | 5 |
| Coco-Caprylate/Caprate | 5 |
| Diisostearoyl Polyglyceryl-3 Dimer Dilinoleate | 1.4 |
| Polyglyceryl-4 Diisostearate/Polyhydroxystearate/Sebacate | 0.8 |
| Glycerin | 5 |
| Citric Acid | 0.09 |
| Sodium Citrate + Aqua | 0.175 |
| Potassium Sorbate | 0.13 |
| Prunus Amygdalus Dulcis Oil | 0.5 |
| Magnesium Sulfate | 0.7 |
| Aqua | ad 100 |

### Example 3: O/W Cream

| | % by |
|---|---|
| INCI | weight |
| Decylene Glycol | 0.1 |
| Propylene Glycol Diheptanoate | 3.5 |
| Levulinic Acid + Aqua + Glycerin + Sodium Levulinate | 0.5 |
| Tocopherol | 0.01 |
| Caprylic/Capric Triglyceride | 3.5 |
| Simmondsia Chinensis Seed Oil | 2 |
| Butyrospermum Parkii Butter | 1.5 |
| Prunus Amygdalus Dulcis Oil | 2 |
| Sodium Stearoyl Glutamate | 0.25 |
| Glyceryl Stearate | 2 |
| Glyceryl Caprylate | 0.2 |
| Parfum | 0.2 |
| Glycerin | 7.5 |
| Cetearyl Alcohol | 3.5 |
| Alcohol | 6 |
| Aqua | ad 100 |

### Example 4: O/W Cream

| | % by |
|---|---|
| INCI | weight |
| Propylene Glycol Diheptanoate | 3.0 |
| Levulinic Acid + Aqua + Glycerin + Sodium Levulinate | 0.5 |
| Ethylhexylglycerin | 0.2 |
| Aloe Barbadensis Leaf Juice Powder | 0.4 |
| Coco-Caprylate/Caprate | 1 |
| Simmondsia Chinensis Seed Oil | 3.5 |
| Butyrospermum Parkii Butter | 0.5 |
| Prunus Amygdalus Dulcis Oil | 3 |
| Dicaprylyl Ether | 1 |
| Sodium Stearoyl Glutamate | 0.3 |
| Glyceryl Stearate | 2 |
| Distarch Phosphate | 1 |
| Glyceryl Caprylate | 0.2 |
| Parfum | 0.2 |
| Glycerin | 7.0 |
| Cetearyl Alcohol | 4.5 |
| Xanthan Gum | 0.3 |
| Alcohol | 4 |
| Aqua | ad 100 |

1.-17. (canceled)

18. A cosmetic product, wherein the product comprises
(a) a packaging of high-density polyethylene (HDPE), low-density polyethylene (LDPE), polypropylene (PP) or polyethylene terephthalate (PET), and
(b) a cosmetic preparation comprising one or more propylene glycol diesters.

19. The cosmetic product of claim 18, wherein (a) is made of high-density polyethylene (HDPE).

20. The cosmetic product of claim 18, wherein (a) has a wall thickness of from 1 mm to 8 mm.

21. The cosmetic product of claim 18, wherein (a) does not have any protective layer on an inner side which faces (b).

22. The cosmetic product of claim 18, wherein (b) comprises one or more of propylene glycol diheptanoate, propylene glycol dicaprate, propylene glycol dicaprylate/dicaprate, propylene glycol dibenzoate.

23. The cosmetic product of claim 18, wherein (b) comprises propylene glycol diheptanoate.

24. The cosmetic product of claim 18, wherein (b) is free of dimethicone.

25. The cosmetic product of claim 18, wherein (b) is free of silicone oil, mineral oil, paraffin wax, microcrystalline wax, shellac wax and polyethylene waxes.

26. The cosmetic product of claim 24, wherein (b) is free of silicone oil, mineral oil, paraffin wax, microcrystalline wax, shellac wax and polyethylene waxes.

27. The cosmetic product of claim 18, wherein (b) is free of polyacrylates, crosslinked acrylate/C10-C30 alkyl acrylate polymers, vinylpyrrolidone/hexadecene copolymers, 3-(4-methylbenzylidene)camphor, 2-hydroxy-4-methoxybenzophenone, 2-ethylhexyl 4-methoxycinnamate, ethylhexyl 2-cyano-3,3-diphenylacrylate, parabens, methylisothiazolinone, chloromethylisothiazolinone, DMDM hydantoin, polyethylene glycol ethers, polyethylene glycol esters.

28. The cosmetic product of claim 26, wherein (b) is free of polyacrylates, crosslinked acrylate/C10-C30 alkyl acrylate polymers, vinylpyrrolidone/hexadecene copolymers, 3-(4-methylbenzylidene)camphor, 2-hydroxy-4-methoxybenzophenone, 2-ethylhexyl 4-methoxycinnamate, ethylhexyl 2-cyano-3,3-diphenylacrylate, parabens, methylisothiazolinone, chloromethylisothiazolinone, DMDM hydantoin, polyethylene glycol ethers, polyethylene glycol esters.

29. The cosmetic product of claim 18, wherein (b) is present in the form of an emulsion.

30. The cosmetic product of claim 18, wherein (b) is present in the form of an oil-in-water emulsion (O/W emulsion).

31. The cosmetic product of claim 18, wherein (b) comprises from 0.1% to 10% by weight of the one or more propylene glycol diesters, based on a total weight of the preparation.

32. The cosmetic product of claim 18, wherein an oil phase of (b) comprises at least 3% by weight of the one or more propylene glycol diesters, based on a total weight of the oil phase.

33. The cosmetic product of claim 18, wherein (b) further comprises one or more of octyldodecanol, caprylic/capric triglyceride, cocoglycerides, tridecyl stearate, tridecyl trimellitate.

34. The cosmetic product of claim 18, wherein (b) further comprises one or more oils selected from polycitronellol, polycitronellol acetate, triolein, ethylhexyl acetoxystearate, acetyl ethylhexyl polyhydroxystearate.

35. The cosmetic product of claim 33, wherein (b) further comprises one or more of polycitronellol, polycitronellol acetate, triolein, ethylhexyl acetoxystearate, acetyl ethylhexyl polyhydroxystearate.

36. The cosmetic product of claim 18, wherein (b) further comprises tocopherol.

37. The cosmetic product of claim 18, wherein (b) further comprises one or more of almond oil, sunflower oil, hydrogenated castor oil.
